(19) United States
(12) Patent Application Publication
(10) Pub. No.: US 2012/0328719 A1
(43) Pub. Date: Dec. 27, 2012

(54) EXTERNAL PREPARATION FOR SKIN

(75) Inventors: Shunsuke Iriyama, Yokohama-shi (JP); Kenichi Umishio, Yokohama-shi (JP); Makoto Tsunenaga, Yokohama-shi (JP); Shinji Inomata, Yokohama-shi (JP)

(73) Assignee: Shiseido Company, Ltd.

(21) Appl. No.: 13/582,588

(22) PCT Filed: Jan. 21, 2011
(86) PCT No.: PCT/JP2011/051113

§ 371 Date: Sep. 4, 2012

(30) Foreign Application Priority Data
Mar. 4, 2010 (JP) 2010-048167

(51) Int. Cl. A61K 36/38

(52) U.S. Cl. 424/730

(57) ABSTRACT

An object of the present invention is to provide prevent or improve decreases in the barrier function and moisture retention function of the skin, and that object is achieved by applying an external preparation for skin that comprises a matrix metalloproteinase inhibitor and a heparanase inhibitor.

### TECHNICAL FIELD

The present invention relates to an external preparation for skin comprising a matrix metalloproteinase (MMP) inhibitor and a heparanase inhibitor, and more particularly, to an external preparation for skin for preventing or improving decreases in the barrier function and moisture retention function of skin.

### BACKGROUND ART

The skin covers the entire body of humans and other animals, and is subjected to the formation of wrinkles, hardening, age spots, darkening and decreased elasticity due to aging as well as external factors such as sunlight, drying, oxidation, environmental stress and psychological stress.

Skin in the natural state is composed of two layers broadly classified as the epidermis and dermis, and a thin, delicate membrane referred to as the epidermal basement membrane is present between the epidermis and dermis. Metabolism of the epidermis is dependent on factors produced by cells of the dermis and blood flow through that pass through this basement membrane, and proliferation and differentiation of the epidermis in the skin are considered to be regulated by the basement membrane and dermis. Thus, communication between the epidermis and dermis mediated by the basement membrane is presumed to play an important role in regulating the functions of the skin epidermis.

In relation thereto, administration of matrix metalloproteinase inhibitor or both matrix metalloproteinase inhibitor and matrix protein production promoter is known promote reformation of skin basement membrane structure (Japanese Unexamined Patent Publication No. 2001-269398), and basement membrane formation promotional effects are further promoted by substances that inhibit serine proteinase as well as substances that enhance production of type IV collagen, type VII collagen or laminin-5, which are the main constituents of epidermal basement membrane components (Japanese Unexamined Patent Publication No. 2004-75661). In addition, compounds that inhibit heparanase activity are known to inhibit wrinkle formation by improving basement membrane function in the body during the course of wrinkle formation (International Publication No. WO 2009/123215).

However, whether or not the barrier function and moisture retention function of the epidermis is significantly improved by a combination of matrix metalloproteinase inhibitor and heparanase inhibitor remains unknown, and due to the extremely complex nature of the interaction between the epidermis and dermis medicated by the basement membrane, the fact that such a combination could significantly improve the barrier function and moisture retention function of the epidermis is extremely unexpected.

### PRIOR ART DOCUMENTS

### Patent Documents

Patent Document 1: Japanese Unexamined Patent Publication No. 2001-269398
Patent Document 2: Japanese Unexamined Patent Publication No. 2004-75661
Patent Document 3: International Publication No. WO 2009/123215

### SUMMARY OF THE INVENTION

### Problems to be Solved by the Invention

An object of the present invention is to provide an external preparation for skin for preventing or improving changes in skin function, and particularly decreases in the barrier function and moisture retention function of the skin.

### Means for Solving the Problems

The inventors of the present invention have found that the barrier function and moisture retention function of the epidermis are significantly improved by a combination of a matrix metalloproteinase (MMP) inhibitor and a heparanase inhibitor.

Thus, the present application includes the inventions indicated below.

[1] An external preparation for skin comprising a matrix metalloproteinase inhibitor and a heparanase inhibitor.
[2] The external preparation for skin described in [1] for preventing or improving decreases in the barrier function and moisture retention function of skin.

### Effects of the Invention

Application of the external preparation for skin comprising a matrix metalloproteinase inhibitor and a heparanase inhibitor to the skin surface is able to significantly increase the amounts of hyaluronic acid and heparan sulfate present in the epidermis of skin as well as normalize differentiation and proliferation of epidermal cells. As a result, the barrier function and moisture retention function of the skin epidermis can be improved.

### BRIEF DESCRIPTION OF THE DRAWINGS

FIG. 1A is a graph indicating hyaluronic acid levels in epidermis in a control group, MMP inhibitor group, heparanase inhibitor group and combination MMP inhibitor+heparanase group.

FIG. 1B is a graph indicating hyaluronic acid levels in culture supernatant in a control group, MMP inhibitor group, heparanase inhibitor group and combination MMP inhibitor+heparanase group.

FIG. 2A is a graph indicating heparan sulfate levels in epidermis in a control group, MMP inhibitor group, heparanase inhibitor group and combination MMP inhibitor+heparanase group.

FIG. 2B is a graph indicating heparan sulfate levels in culture supernatant in a control group, MMP inhibitor group, heparanase inhibitor group and combination MMP inhibitor+heparanase group.

FIG. 3 is a graph indicating the moisture transpiration rate constants of water, 5% glycerin and various types of 0.2% glycosaminoglycans (hyaluronic acid, keratan sulfate, heparan sulfate, chondroitin sulfate A and chondroitin sulfate B).

FIG. 4 indicates photographs indicating immunostaining of perlecan and heparan sulfate in epidermis in a control group, MMP inhibitor group, heparanase inhibitor group and combination MMP inhibitor+heparanase group.

FIG. 5 indicates photographs indicating immunostaining of loricrin and filaggrin in epidermis in a control group, MMP inhibitor group, heparanase inhibitor group and combination MMP inhibitor+heparanase group.

FIG. 6 indicates photographs indicating immunostaining of proliferative cells positive for Ki67 in a control group, MMP inhibitor group, heparanase inhibitor group and combination MMP inhibitor+heparanase group on days 4 and 8 of culturing.

### EMBODIMENTS OF THE INVENTION

Approximately 70% of the human body is composed of water, and in order to perform biological activities in a dry atmosphere, a barrier function is essential for preventing transpiration of moisture present in the body as well as infiltration of foreign matter from the outside world. In addition, a moisture retention function is extremely important for retaining moisture in the body, and the skin is able to contain a suitable level of moisture to maintain flexibility and moistness as a result of these functions.

In normal skin in the natural state, new cells continuously proliferate at a constant rate due to cell division in the basal layer of the epidermis that lies farthest to the inside thereof, and these basal cells are pushed upward and differentiate into prickle cells, granular cells and horny cells, and then finally slough off in the form of skin particles. However, in the case an abnormality occurs in the higher structure of the epidermis due to some cause, this keratinization process of epidermal cells does not proceed normally, and this is thought to cause decreases in the moisture retention function and barrier function of the epidermis.

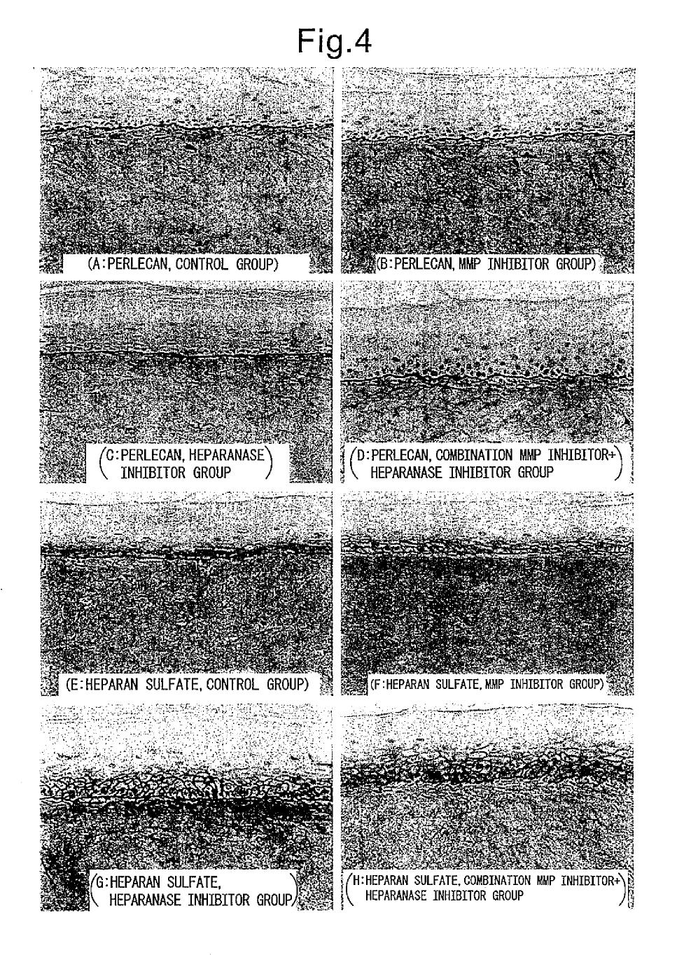

When the inventors of the present invention measured hyaluronic acid levels and heparan sulfate levels present in the epidermis of respective skin models consisting of artificial skin formed in a culture fluid containing a solvent (control group), artificial skin formed in a culture fluid containing N-hydroxy-2-[[(4-methoxyphenyl)sulfonyl](3-picolyl)amino]-3-methylbutaneamide hydrochloride (MMP inhibitor group), artificial skin formed in a culture fluid containing 1-[4-(1H-benzoimidazol-2-yl)-phenyl]-3-[4-(1H-benzoimidazol-2-yl)-phenyl]urea (heparanase inhibitor group) and artificial skin formed in a culture fluid containing N-hydroxy-2-[[(4-methoxyphenyl)sulfonyl] (3-picolyl)amino]-3-methylbutaneamide hydrochloride and 1-[4-(1H-benzoimidazol-2-yl)-phenyl]-3-[4-(1H-benzoimidazol-2-yl)-phenyl]urea (MMP inhibitor+heparanase inhibitor group), both hyaluronic acid and heparan sulfate levels were found to have increased in the MMP inhibitor+heparanase inhibitor group. This means that the combination of MMP inhibitor and heparanase inhibitor has an action that significantly inhibits the decomposition of hyaluronic acid and heparan sulfate in the epidermis. In addition, this inhibitory action on decomposition demonstrated by the combination of MMP inhibitor and heparanase inhibitor can also be confirmed from the conspicuous presence of MMP inhibitor and heparanase inhibitor in the epidermis as determined by immunostaining of heparan sulfate and perlecan, a heparan sulfate proteoglycan (see FIG. 4).

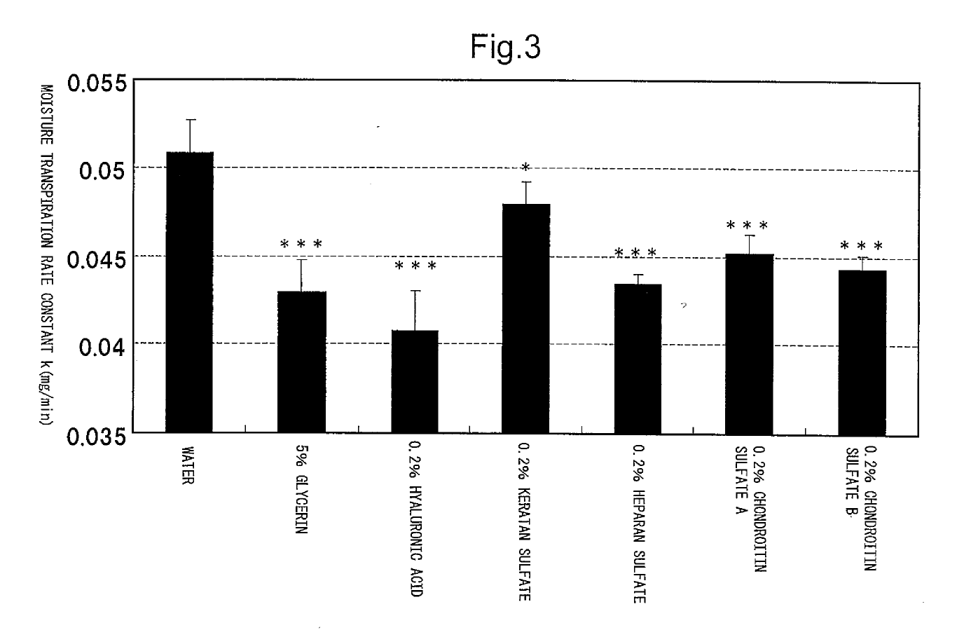

Hyaluronic acid and heparan sulfate are types of glucosaminoglycans, and are known to be widely present on the cell surfaces of mammals. As shown in FIG. 3, since the moisture transpiration rate constants of hyaluronic acid and heparan sulfate are remarkably low among the various types of glucosaminoglycans, hyaluronic acid and heparan sulfate have been determined to possess potent moisture retention action. Thus, the combination of MMP inhibitor and heparanase inhibitor is thought to be able to significantly improve the moisture retention function of the skin.

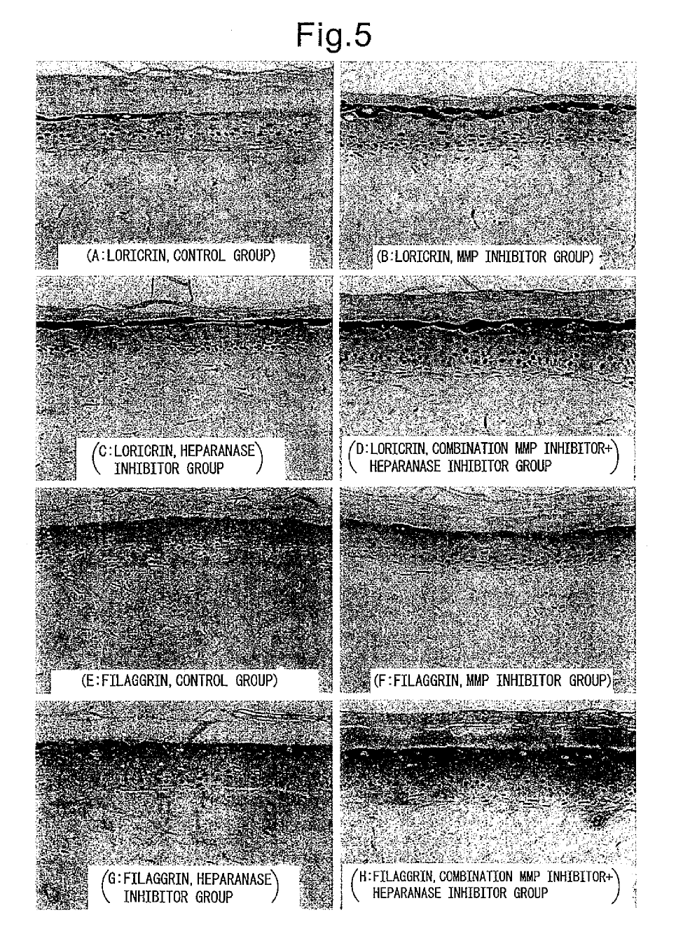

More surprisingly, the inventors of the present invention found that a combination of MMP inhibitor and heparanase inhibitor enables the epidermis structure of artificial skin to be restructured to a structure that is extremely close to that of normal natural skin. More specifically, as shown in FIG. 5, in an artificial skin model of an MMP inhibitor+heparanase inhibitor group, the expressed amounts of known epidermal molecular markers in the form of loricrin and filaggrin increased significantly, and these molecular markers were present not only in basement membrane but were also uniformly present in the granular layer of the epidermis. In normal natural skin, loricrin and filaggrin are thought to be involved in the barrier function of skin by functioning as proteins that bundle keratin present in the granular layer of the epidermis. Thus, the fact that significant amounts of loricrin and filaggrin are uniformly present in the granular layer in a skin model of an MMP inhibitor+heparanase inhibitor group is important evidence that a combination of MMP inhibitor and heparanase inhibitor significantly improves the barrier function of skin.

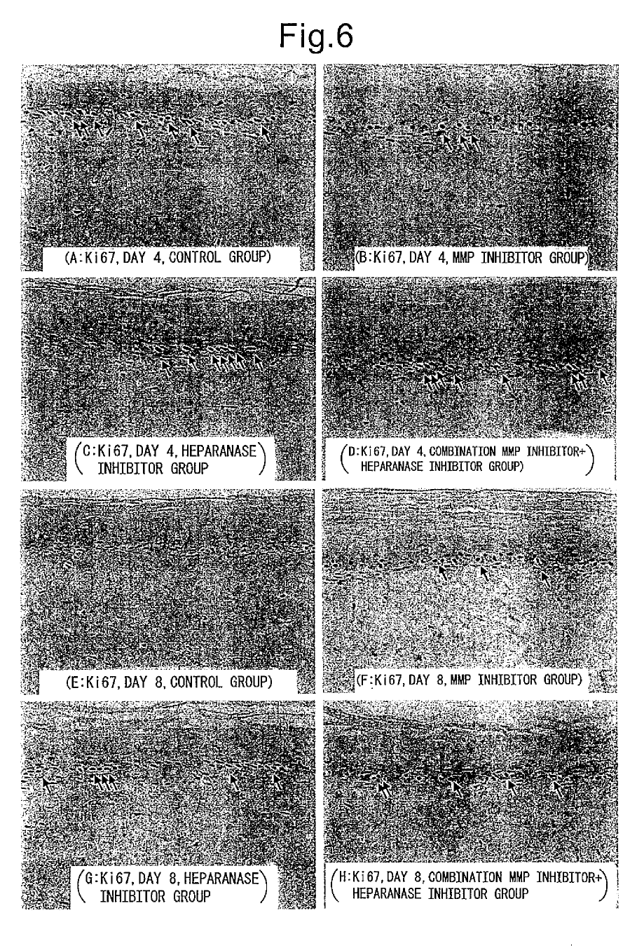

Moreover, in a skin model of an MMP inhibitor+heparanase inhibitor group, proliferative cells positive for Ki67 continued to be present at a high rate even on day 8 of culturing (see FIG. 6). This indicates that proliferation of basal cells of the epidermis is maintained.

Thus, in the present invention, a combination of active ingredients consisting of MMP inhibitor and heparanase inhibitor can be used medically or aesthetically to prevent or improve changes in skin function, and particularly decreases in the barrier function and moisture retention function of skin.

Matrix Metalloproteinase (MMP) Inhibitor

There are no particular limitations on the matrix metalloproteinase inhibitor used in the present invention provided it is a substance that demonstrates inhibitory activity against matrix metalloproteinases. Examples of matrix metalloproteinases include gelatinase, collagenase, stromelysin and matrilysin. Thus, a substance that inhibits gelatinase, collagenase, stromelysin or matrilysin, for example, can be selected for use as an MMP inhibitor.

Specific examples of matrix metalloproteinase inhibitors include Substance CGS27023A (N-hydroxy-2-[[(4-methoxyphenyl)sulfonyl](3-picolyl)amino]-3-methylbutaneamide hydrochloride) (J. Med. Chem. 1997, Vol. 40, p. 2525-2532), and MMP Inhibitor (p-NH₂-Bz-Gly-Pro-D-Leu-Ala-NHOH) (FN-437) (BBRC 1994, Vol. 199, p. 1442-1446). The MMP inhibitor is preferably Substance CGS27023A.

Moreover, various plant extracts and purified products obtained there from can also be used for the matrix metalloproteinase inhibitor of the present invention. Examples of plant extracts include extracts of such plants include Thymus serpyllum L., Valeriana fauriei Briquet and similar plants thereof (Valerianaceae), Diospyros kaki Thunberg (Ebenaceae), Astragalus sinicus Linne, Crataegus cuneata Siebold et Zuccarini (Rosaceae), Paeonia suffructicosa Andrews (Poeinia montan Sims) (Paconiaceae), Thea sinensis Linne var. assamica Pierre (Thcaccae), Eucalyptus globulus Labillardiere and similar plants thereof (Myrtaceae), Potentilla tormentilla Schrk (Rosaceae), Tilia cordata Mill., Tilia platyphyllus Scop., Tilia europaea Linne (Tiliaceae), Betula alba Linne (Betulaceze), Origanummajorana L., Uncaria gambir Roxburgh (Rubiaceae), Juglans regia Linne var. sinensis De Candolie and similar plants thereof (Juglandaceae), Sophora flavescens Aiton (Leguminoseae), Sangulsorba officinalis Linne (Rosaceae), Hypericum perforatum Linne or Hypericum erectum Thunberg (Guttiferae), Thea sinensis Linne (Theaceae), Curcuma longa L (Zingiberaceae), refined Curcuma longa L in the form of curcumin, Symplocos racemosa, Cyperus rotundus, Cyperus scariosus, Gaultheria fragrantissima, Acacia fornensia, Terminalia chebula, Flous bengalensis, Cassia fistula Linn, Lyonia ovalifolia, Calophyllum inophyllum, Ficus religiosa, Potentilla tormentilla S., Persea americana Mill, Garcinia mangostana L., Cocos balsamifera (L) D C., and Cinnamomum cassia Bl.

These plant extracts are obtained from extracts of the root, leaves, stems or flowers in the case of herbaceous plants, or from the root, buds, bark, fruit, leaves or flowers in the case of woody plants. Extracts from these plants are obtained by drying the plant material as necessary and then macerating or crushing as necessary, followed by extracting with an aqueous extraction solvent or organic solvent. Examples of aqueous extraction solvents that can be used include cold water, warm water or hot water at a temperature equal to the boiling point or lower, while examples organic solvents include methanol, ethanol, 1,3-butanediol and ether, and these can be used at normal temperature or after heating.

Heparanase Inhibitor

There are no particular limitations on the heparanase inhibitor used in the present invention provided it is a substance that demonstrates inhibitory activity against heparanase. Heparanase is an enzyme present in various cells that specifically decomposes the heparan sulfate chains of various heparan sulfate proteoglycans. In the skin, it is produced by epidermal keratinocytes that compose the epidermis as well as dermal fibroblasts and vascular endothelial cells. Production is known to increase in various cancer cells, and it has been suggested to be related to cancer malignancy. High production levels of heparanase in cancer cells are known to indicate a high potential for metastasis as well as a high likelihood of induction of vascular neogenesis (see Vlodaysky, I. et al., Semin. Cancer Biol. 2002; 12(2): 121-129).

Specific examples of heparanase inhibitors include 4-1H-benzoimidazol-2-yl-phenylamine and derivatives thereof, cinnamic acid derivatives such as (E)-N-(5-methylisoxazolyl-3-yl)-3-(3,4,5-trimethoxyphenyl)acrylamide or (E)-3-(2-chlorophenyl)-N-(pyridin-3-ylmethyl)acrylamide, tetrazole derivatives, naphthalene derivatives, cycloalkanone derivatives, 4-alkylresorcinols such as 4-isobutylresorcinol, or 1-[4-(1H-benzimidazol-2-yl)-phenyl]-3-[4-(1H-benzimidazol-2-yl)-phenyl]urea.

Moreover, various plant extracts and purified products obtained there from can also be used for the heparanase inhibitor of the present invention. Examples of such plant extracts include Valeriana extract obtained from Valeriana fauriei Briquet or similar plants thereof (Valerianaceae), cypress extract obtained from Chamaecyparis such as Chamaecyparis obtusc, Chamaecyparis oatuse var. formosana, Thujupsis delabrata or a variety thereof (T.d. var. hondae), kiwi extract obtained from A. cninensis, lemon extract obtained from C. limon, tomato extract obtained from L. esculentum, garlic extract obtained from A. sativum, lily extract obtained from Lilium such as L. candidum, peucedanum extract obtained from P. japonicum, angelica root extract obtained from C. aurantium, soapberry extract obtained from S. mukorossi, parsley extract obtained from P. crispum, jujube extract obtained from Z. jujuba var. inermis, tangerine peel extract obtained from C. unshiu or its related species, C. chachiensis, and nettle extract obtained from U. thunbergiana.

As was previously described, these plant extracts can also be extracted using methods commonly employed in the art.

In the present invention, a combination of one or more matrix metalloproteinase (MMP) inhibitors and one or more heparanase inhibitors can be used as active ingredients. These can be in the form of an aqueous solutions, oily liquids, other solutions, milky lotions creams, gels, suspensions, microcapsules, powders, granules, capsules or solids and the like. After preparing these active ingredients in these forms using a known method, they can be applied, affixed, sprayed, injected, drank or inserted into the body in the form of various preparations such as a lotion, milky lotion, cream, ointment, plaster, poultice, aerosol, injection, internal medication (such as a tablet, powder, granules, pill, syrup or troche) or suppository. Among these preparations, external preparations for skin such as lotions, milky lotions, creams, ointments, plasters, poultices or aerosols are considered to be preferable forms. A preparation referred to here, and particularly an external preparation for skin, includes pharmaceuticals, quasi drugs and cosmetics, and is used with the same meaning hereinafter. Although varying according to the type thereof, the amount of MMP inhibitor contained in the external preparation for skin of the present invention is typically about 10 μg/L to 10 g/L, preferably about 100 μg/L to 1 g/L and most preferably about 1 mg/L to 100 mg/L. Although varying according to the type thereof, the amount of heparanase inhibitor contained in the external preparation for skin of the present invention is typically about 10 μg/L to 100 g/L, preferably about 100 μg/L to 10 g/L, and most preferably about 1 mg/L to 1 g/L.

Vehicles or fragrances and the like ordinarily used during the preparation thereof, as well as oils, surfactants, antiseptics, chelating agents, water-soluble polymers, alcohols, thickeners, powdered components, ultraviolet absorbers, moisturizers, medicinal components, antioxidants, neutralizers, pH regulators, cleansers, desiccants or emulsifiers and the like can be suitably incorporated in the external preparation for skin of the present invention. In the case of incorporating each of these components in the external preparation for skin of the present invention, it is necessary that they be incorporated within a range that does not impair the desired effects of the present invention.

Among the arbitrarily incorporated components suitably incorporated as described above, examples of oily components include higher alcohols in the manner of linear alcohols such as lauryl alcohol, cetyl alcohol, stearyl alcohol, myristyl alcohol or oleyl alcohol and branched alcohols such as glycerin monostearyl ether, lanolin alcohol, cholesterol, phytosterol or isostearyl alcohol, higher fatty acids such as lauric acid, myristic acid, palmitic acid or stearic acid, waxes such as solid paraffin, beeswax, hydrogenated castor oil, carnauba wax or Bareco wax, animal and plant oils and fats such as beef tallow, pork tallow, mutton tallow, squalane, coconut oil, palm oil, palm kernel oil, soybean oil, olive oil, cottonseed oil, jojoba oil, castor oil or lanolin, mineral oils such as liquid paraffin or vaseline, and synthetic oils such as trimethylpropane triisostearate, isopropyl myristate, glycerol tri-2-ethylhexanoate, pentaerythritol tetra-2-ethylhexanoate, silicone oil or polyoxyethylene (POE)-polyoxypropylene (POP) pentaerythritol ether.

Examples of surfactants include anionic surfactants in the manner of fatty acid soaps such as soap materials, sodium laurate or sodium palmitate, higher alkyl sulfates such as sodium lauryl sulfate or potassium lauryl sulfate, alkyl ether sulfates such as triethanolamine POE lauryl ether sulfate or sodium POE lauryl ether sulfate, N-acyl sarcosinates such as sodium lauroyl sarcosinate, higher fatty acid amidosulfonates such as sodium N-myristyl-N-methyltaurate or sodium coconut oil fatty acid methyl tauride, phosphate ester salts such as POE-stearyl ether phosphate, sulfosuccinates such as sodium monolauroyl monoethanolamide POE sulfosuccinate or propylene glycol sodium lauryl sulfosuccinate, alkylbenzene sulfonates such as linear sodium dodecylbenzene sulfonate or linear triethanolamine dodecylbenzene sulfonate, N-acylglutamates such as disodium N-stearoyl glutamate or monosodium N-lauroyl glutamate, higher fatty acid ester sulfate ester salts such as sodium hydrogenated palm oil fatty acid glycerin sulfate, sulfonated oils such as turkey red oil, POE-alkyl ether carboxylic acids, POE-alkyl allyl ether carboxylates, α-olefin sulfonates, higher fatty acid ester sulfonates, secondary alcohol sulfate ester salts, higher fatty acid alkyloyl amide sulfate ester salts, sodium lauroyl monoethanol amide succinates and sodium caseinate; cationic surfactants in the manner of alkyl trimethyl ammonium salts such as stearyl trimethyl ammonium chloride or lauryl trimethyl ammonium chloride, dialkyl dimethyl ammonium salts such as distearyl dimethyl ammonium chloride, alkyl pyridinium salts such as cetyl pyridinium chloride, alkyl quaternary ammonium salts, alkyl dimethyl benzyl ammonium salts, alkyl isoquinolinium salts, dialkyl morphonium salts, POE-alkyl amines, alkyl amine salts, polyamine fatty acid derivatives, amyl alcohol fatty acid derivatives or benzalkonium chloride; amphoteric surfactants in the manner of imidazoline-based amphoteric surfactants such as sodium 2-cocoyl-2-imidazolinium hydroxide-1-carboxyethyloxy disodium salt or betaine-based surfactants such as amide betaines or sulfobetaines; lipophilic nonionic surfactants in the manner of sorbitan fatty acid esters such as sorbitan monooleate, sorbitan monoisostearate, sorbitan monolaurate, sorbitan monopalmitate or sorbitan trioleate, glycerin polyglycerin fatty acid esters such as cottonseed oil fatty acid monoglycerides, glyceryl monostearate, glyceryl sesquioleate or glyceryl monostearate/malate, propylene glycol fatty acid esters such as propylene glycol monostearate, hydrogenated castor oil derivatives, glycerin alkyl ethers and POE-methylpolysiloxane copolymers; and, hydrophilic nonionic surfactants in the manner of POE-sorbitan fatty acid esters such as POE-sorbitan monooleate or POE-sorbitan monostearate, POE-sorbitol fatty acid esters such as POE-sorbitol monolaurate, POE-sorbitol monooleate or POE-sorbitol monostearate, POE-glycerin fatty acid esters such as POE-glycerin monooleate or POE-glycerin distearate, POE-fatty acid esters such as POE-monooleate, POE-distearate or POE-monodioleate, POE-alkyl ethers such as POE-lauryl ether, POE-oleyl ether or POE-cholestanol ether, POE-alkyl phenyl ethers such as POE-octyl phenyl ether or POE-nonyl phenyl ether, pluronic-type surfactants such as Pluronic, POE/POP-alkyl ethers such as POE/POP-monobutyl ether, POE/POP-cetyl ether or POE/POP-glycerol ether, POE-castor oil derivatives or POE-hydrogenated castor oil derivatives such as POE-castor oil, POE-hydrogenated castor oil, POE-hydrogenated castor oil monoisostearate or POE-hydrogenated castor oil maleate, POE-beeswax-lanolin derivatives such as POE-sorbitol beeswax, alkanol amides such as palm oil diethanolamide or fatty acid isopropanolamide, POE-propylene glycol fatty acid esters, POE-fatty acid amides, POE-alkyl amines, sucrose fatty acid esters and alkylethoxydimethylamine oxides.

Examples of alcohols include lower alcohols such as ethanol, propanol or isopropanol.

Examples of thickeners include water-soluble polymers in the manner of plant-based polymers such as gum arabic, tragacanth gum, galactan, carob gum, guar gum, carrageenan, pectin, agar or starch (including cornstarch, potato starch, wheat starch and rice starch), microbial polymers such as dextran or pullulan, starch-based polymers such as carboxymethyl starch or methyl hydroxypropyl starch, animal-based polymers such as collagen, casein or gelatin, cellulose-based polymers such as methyl cellulose, nitrocellulose, ethyl cellulose, hydroxyethyl cellulose, sodium cellulose sulfate, hydroxypropyl cellulose, carboxymethyl cellulose or crystalline cellulose, alginic acid-based polymers such as sodium alginate or propylene glycol alginate, vinyl-based polymers such as polyvinyl methyl ether or carboxyvinyl polymer, POE-based polymers, POE/POP-based copolymers, acrylic-based polymers such as sodium polyacrylate or polyacrylamide, polyethyleneimine, cationic polymers, and inorganic water-soluble polymers such as bentonite, magnesium aluminum silicate, laponite, hectorite and silicic anhydride.

Examples of chelating agents include citramalic acid, agaric acid, glyceric acid, shikimic acid, hinoki thiol, gallic acid, tannic acid, caffeic acid, ethylenediamine tetraacetate, ethylene glycol diamine tetraacetate, diethylenetriamine pentaacetate, phytic acid, polyphosphoric acid, metaphosphoric acid, derivatives thereof, alkali metal salts thereof and carboxylic acid esters thereof.

Examples of ultraviolet absorbers include benzoic acid-based ultraviolet absorbers such as paraminobenzoic acid; anthranylic acid-based ultraviolet absorbers such as methyl anthranilate; salicylic acid-based ultraviolet absorbers such as octyl salicylate; and cinnamic acid-based ultraviolet absorbers such as isopropyl-p-methoxycinnamate or octyl-p-methoxycinnamate.

Examples of moisturizers include polyethylene glycol, propylene glycol, dipropylene glycol, 1,3-butylene glycol, glycerin, diglycerin, xylitol, maltitol, maltose, D-mannitol, glucose, fructose, sodium chondroitin sulfate, sodium hyaluronate, sodium lactate, glucosamine and cyclodextrin.

Examples of medicinal components include vitamins such as vitamin A, retinol, retinol palmitate, pyridoxine hydrochloride, benzyl nicotinate, nicotinic amide, dl-α-tocopherol nicotinate, magnesium ascorbyl phosphate, vitamin D2, dl-α-tocopherol, pantothenic acid or biotin; antiinflammatory agents such as glycyrrhizinic acid; whitening agents such as arbutin, potassium 4-methoxysalicylate or 2-O-ethylascorbic acid or glucoside ascorbate; hormones such as estradiol; astringents such as zinc oxide or tannic acid, refreshing agents such as L-menthol or camphor; and, lysozyme chloride, pyridoxine hydrochloride and sulfur. Moreover, various types of extracts can be incorporated that exhibit diverse pharmacological effects, examples of which include Houttunia cordata extract, Phellodendron bark extract, licorice root extract, paeony root extract, moutan bark extract, sponge gourd extract, saxifrage extract, eucalyptus extract, clove extract, chamomile extract, seaweed extract and thyme extract.

Examples of antiseptics include parahydroxybenzoic acid esters such as methyl parahydroxybenzoate, ethyl parahydroxybenzoate or butyl parahydroxybenzoate, benzoic acid, salicylic acid, sorbic acid, parachlorometacresol, hexachlorophene, benzalkonium chloride, chlorhexidine chloride, trichlorocarbanilide, photosensitive pigment, phenoxyethanol and isomethylthiazolinone.

Examples of neutralizers include 2-amino-2-methyl-1-propanol, 2-amino-2-methyl-1,3-propanediol, sodium hydroxide, potassium hydroxide, triethanolamine and sodium carbonate.

Examples of pH regulators include lactic acid, citric acid, glycolic acid, succinic acid, tartaric acid, malic acid, sodium bicarbonate and ammonium bicarbonate.

Examples of antioxidants include ascorbic acid, α-tocopherol and carotinoids.

The aforementioned components are exemplary and are not intended to be limiting. In addition, these components can be suitably combined and incorporated in accordance with formulations corresponding to the desired form.

Furthermore, medicinal components can be incorporated over a wide range provided they do not impair the desired effects of the present invention. The external preparation for skin of the present invention prepared in this manner is able to prevent or improve decreases in skin function, and particularly decreases in the barrier function and moisture retention function of skin. Thus, the external preparation of skin of the present invention is effective against diseases or symptoms caused by decreases in the barrier function and moisture retention function of the skin, examples of which include contact dermatitis, allergic dermatitis, atopic dermatitis, dry skin, sensitive skin, oily skin, acne, burns, sunburn, age spots, wrinkles and skin aging.

In addition, another embodiment of the present invention provides an artificial skin culture fluid comprising a matrix metalloproteinase inhibitor and a heparanase inhibitor.

An arbitrary culture medium conventionally used to produce artificial skin can be used for the basal medium used to produce artificial skin, and examples of these media include Dulbecco's modified Eagle's medium (DMEM) containing 10% fetal bovine serum, DMEM-Ham's F12 (3:1) medium containing 10% fetal bovine serum, 5 μg/ml of transferrin, 5 μg/ml of insulin, 2 nM triiodothyronine, 0.1 nM of cholera toxin and 0.4 μg/ml of hydrocortisone, and medium obtained by mixing keratinocyte growth medium (KGM) and DMEM containing 10% fetal bovine serum at a ratio of 1:1. Although varying according to the type thereof, the amount of MMP inhibitor added to these basal media is typically about 10 μg/L to 10 g/L, preferably about 100 μg/L to 1 g/L, and most preferably about 1 mg/L to 100 mg/L. In addition, although varying according to the type thereof, the amount of heparanase inhibitor added to these basal media is typically about 10 μg/L to 100 g/L, preferably about 100 μg/L to 10 g/L, and most preferably about 1 mg/L to 1 g/L.

The previously described components ordinarily used to prepare culture fluid can also be suitably incorporated in the artificial skin culture fluid of the present invention. In the case of incorporating each of these components into the artificial skin culture fluid of the present invention, the components are required to be incorporated within a range that does not impair the desired effects of the present invention.

In producing artificial skin, a dermis model is first prepared. A dermis model may contain type I collagen gel containing human fibroblasts, chitin, chitosan, chondroitin sulfate and human fibroblasts crosslinked with collagen in an intermediate or upper portion thereof, or may contain collagen gel containing human fibroblasts in an intermediate or upper portion thereof after having been compressed by centrifugation and the like. Collagen gel can be prepared, for example, in the manner described below. The collagen gel is prepared by preparing a collagen solution in which fibroblasts have been suspended on ice followed by gelling the collagen in a Petri dish. Subsequently, the gel is separated from the walls of the Petri dish and the collagen gel is allowed to contract in a CO₂ incubator. In addition, in order to allow the collagen gel to accurately mimic the skin dermis, vascular endothelial cells, fat cells and nerve cells can be contained in addition to the fibroblasts.

Next, epidermal cells such as human normal epidermal keratinocytes are cultured on the aforementioned dermis model to form epidermis. Formation of an epidermal layer by culturing epidermal cells can be carried out in the manner described below. First, the dermis model is placed on a metal mesh and the like or is allowed to stand undisturbed in a cell culture insert. Moreover, a glass ring is placed over the dermis model and a suspension of human-derived epidermal keratinocytes is placed in the glass ring so as to prevent leakage. Alternatively, the dermis model is adhered to the inner walls of the cell culture insert and a suspension of epidermal keratinocytes is added so as to overflow over the upper portion of the dermis model. The keratinocytes are then allowed to adhere in a CO₂ incubator, and in the case of using a ring, culturing may be carried out after removing the ring or while continuing to leave the ring in place. A rubber ring may also be used instead of a glass ring. In addition, in order to allow the dermis model to more accurately mimic human epidermis, pigment cells and Langerhans cells may also be added in addition to the epidermal cells. The aforementioned culture medium is filled to the boundary of the epidermal layer, and culturing is continued while exposing the epidermal layer to air to form a horny layer.

According to this method, artificial skin can be obtained that has a structure that is extremely close to the epidermis structure of normal natural skin. In addition, as shown in FIG. 6, in a skin model of an MMP inhibitor+heparanase inhibitor group, since proliferative cells positive for Ki67 continued to be present at a high ratio even on day 8 of culturing, the artificial skin formed with the culture fluid of the present invention is thought to enable culturing for a long period of time.

In the case natural skin of the body has been subjected to a lesion or injury due to some cause, the artificial skin of the present invention obtained in this manner can be clinically transplanted for use as an alternative thereof. In addition, since the artificial skin of the present invention corrects keloid scars caused by burns, skin graft scars, surgical scars, deep wrinkles, deep wound scars, pimple scars, large hair follicles or fine wrinkles and the like, it can also be applied aesthetically to surface irregularities on the skin. Moreover, the artificial skin of the present invention can also be used as a research or testing model to conduct skin permeability tests or testing of efficacy or toxicity of pharmaceuticals or cosmetics, or conduct research on wound healing, chemotaxis, infiltration of cancer cells, metastasis of cancer cells or the progression of cancer and the like.

### EXAMPLES

### 1. Skin Model Culturing Method

A skin model (EFT-400, MatTek Corp.) was cultured in a special-purpose medium (EFT-400-ASY, MatTek Corp.). Dimethylsulfoxide (DMSO) and ethanol were added to the special-purpose medium to a final concentration of 0.1% for use as a control group, 50 mM 1-[4-(1H-benzoimidazol-2-yl)-phenyl]-3-[4-(1H-benzoimidazol-2-yl)-phenyl]urea (solvent: DMSO) were added to the special-purpose medium to a final concentration of 50 μM for use as a heparanase inhibitor group, 10 mM N-hydroxy-2-[[(4-methoxyphenyl)sulfonyl](3-picolyl)amino]-3-methylbutaneamide hydrochloride (CGS27023A, solvent: ethanol) were added to the special-purpose medium to a final concentration of 10 μM for use as an MMP inhibitor group, and 50 mM 1-[4-(1H-benzoimidazol-2-yl)-phenyl]-3-[4-(1H-benzoimidazol-2-yl)-phenyl]urea (solvent: DMSO) and 10 mM CGS27023A (solvent: ethanol) were added to the special-purpose medium to final concentrations of 50 μM and 10 μM, respectively, for use as a combination heparanase inhibitor+metalloproteinase (MMP) inhibitor group. The medium was replaced daily at 2 mL/well, and the skin model was recovered and used for experimentation on days 4 and 8 of culturing. In addition, during replacement of the medium, all of the culture fluid was collected and stored at −80° C.° for use in the experiments described below.

### 2. Measurement of Hyaluronic Acid Levels

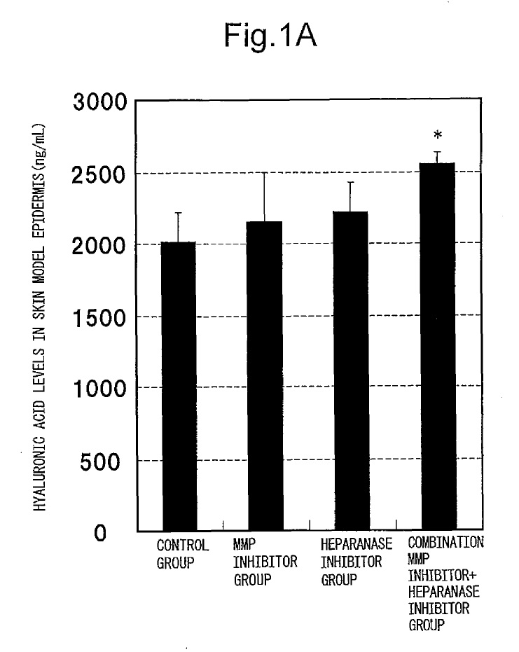

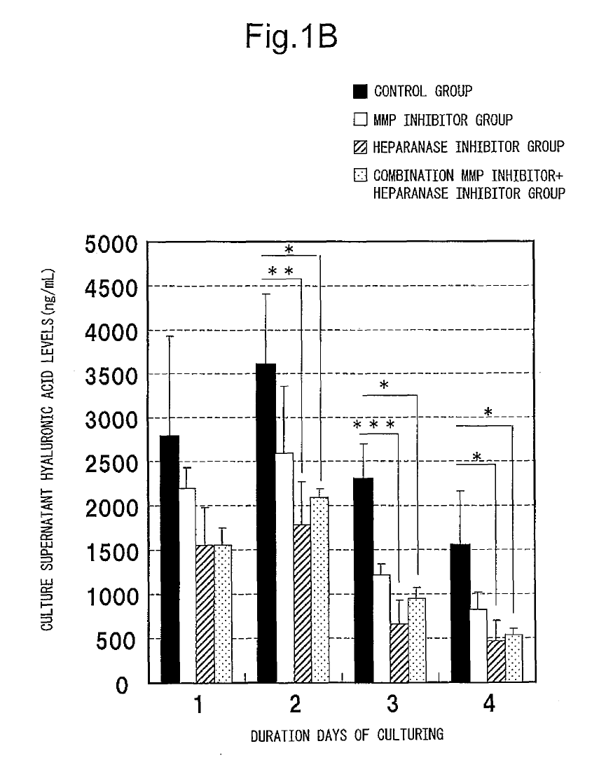

Measurement of hyaluronic acid levels was carried out on the aforementioned cultured control group (n=5), MMP inhibitor group (n=5), heparanase inhibitor group (n=5) and combination MMP inhibitor+heparanase inhibitor group (n=5) (n refers to the number of each skin model used in testing). Each skin model was solubilized in LIPA buffer (Nacalai Tesque Inc.) after separating the epidermis and dermis, and supernatant obtained by removing the insoluble fraction by centrifugation was used as the epidermis fraction of the skin models. The culture supernatants stored at −80° C. were then thawed and hyaluronic acid levels in these solutions were then measured with a hyaluronic acid assay kit (Seikagaku Corp.). In the epidermis of the skin models, a significant increased in hyaluronic acid levels were able to be confirmed in the epidermis of the combination matrix metalloproteinase (MMP) inhibitor+heparanase inhibitor group (FIG. 1A). On the other hand, significant decreases in hyaluronic acid levels in the culture supernatant were able to be confirmed in the group containing heparanase inhibitor (FIG. 1B). On the basis of these results, the presence of both MMP inhibitor and heparanase inhibitor was clearly determined to inhibit decomposition of hyaluronic acid and increase hyaluronic acid levels in the epidermis.

### 3. Measurement of Heparan Sulfate Levels

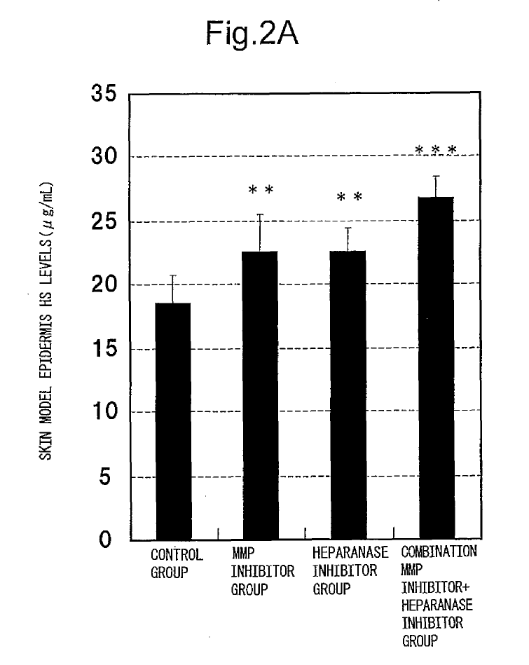

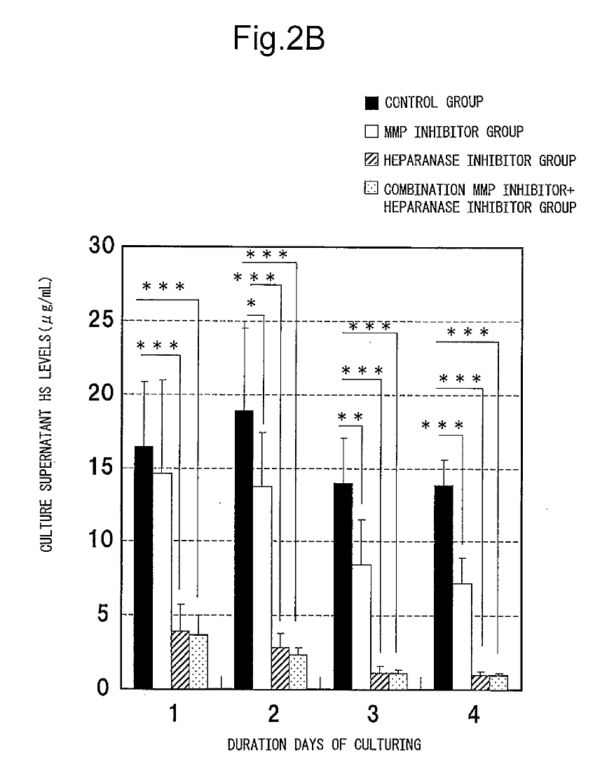

Heparan sulfate levels were measured in a control group (n=5), MMP inhibitor group (n=5), heparanase inhibitor group (n=5) and combination MMP inhibitor+heparanase inhibitor group (n=5) in the same manner as during measurement of hyaluronic acid levels (n refers to the number of each skin model used in testing). Heparan sulfate levels in the culture supernatant and epidermis fractions of the skin models were measured with a heparan sulfate assay kit (Seikagaku Corp.). According to the results of the levels of heparan sulfate in the culture supernatant, heparan sulfate levels decreased significantly in the combination MMP inhibitor+heparanase inhibitor group as compared with the control group (FIG. 2B). In addition, measurement of heparan sulfate levels in the epidermis of the skin models revealed a significant increase in heparan sulfate levels in the combination MMP inhibitor+heparanase inhibitor group as compared with the control group (FIG. 2A). On the basis of these results, in those groups containing heparanase inhibitor, and particularly in the combination MMP inhibitor+heparanase inhibitor group, decomposition of heparan sulfate was inhibited and heparan sulfate levels in the epidermis were clearly determined to increase.

### 4. Measurement of Moisture Transpiration Rate Constants

Heparan sulfate (n=5), chondroitin sulfate A (n=5), chondroitin sulfate B (n=5), keratan sulfate (n=5) and hyaluronic acid (n=5) were dissolved in MilliQ to prepare 0.2% aqueous solutions of each, and 5.0% glycerin (n=5) and MilliQ were prepared for use as a comparative sample (n refers to the respective number of samples). Filter paper cut to a size of 2 cm×2 cm was placed on a balance, and the filter paper was impregnated with 10 μl of each solution followed by measuring the weight of the filter paper for 8 minutes at 1 minute intervals. The weight (mg) of the filter paper from 3 to 8 minutes after the start of measurement was plotted on the vertical axis, elapsed time (min) was plotted on the horizontal axis, and the slope (mg/min) of the resulting line was calculated as the moisture transpiration rate constant (FIG. 3). Among these graphs, although the moisture transpiration rate constant for 0.2% hyaluronic acid was remarkably low, the moisture transpiration rate constants for 0.2% heparan sulfate and 5% glycerin were roughly the same, thereby demonstrating inhibition of moisture transpiration.

### 5. Immunostaining of Skin Models

The skin models following culturing were cut in half with a scalpel and placed in acetone at 4° C. followed by allowing to stand undisturbed for 48 hours at 4° C. The solution was replaced with acetone, methyl benzoate and xylene in that order followed by embedding in paraffin to prepare paraffin blocks in accordance with the AMeX method. Tissue sections having a thickness of 3 μm were prepared followed by immunostaining with perlecan (FIGS. 4A to 4D), heparan sulfate (FIGS. 4E to 4H), loricrin (FIGS. 5A to 5D), filaggrin (FIGS. 5E to 5H) and Ki67 (FIG. 6) on days 4 and 8 of culturing. The resulting immunostaining images are shown in FIGS. 4 to 6. Although expression levels of heparan sulfate, filaggrin and loricrin increased in the heparanase inhibitor group, MMP inhibitor group and combination MMP inhibitor+heparanase inhibitor group in comparison with the control group, the increase was particularly remarkable in the combination MMP inhibitor+heparanase inhibitor group. In addition, in the groups containing heparanase inhibitor, the presence of proliferative cells positive for Ki67 was clearly determined to be maintained.

### 6. Gene Extraction

The skin models that had been cut in half after culturing were promptly separated into epidermis and dermis with a forceps. Each sample was placed in 350 μL of RLT buffer (RNeasy Mini Kit, Qiagen Corp.) followed by the addition of zirconia balls and pulverizing the tissue with a pulverizer. Subsequently, RNA was extracted with the RNeasy Mini Kit (Qiagen Corp.). Moreover, The extracted RNA was amplified after labeling with Cy3 using the Quick Amp-Labeling Kit (Agilent Corp.) followed by carrying out a total human microarray assay. Those gene groups that demonstrated fluctuations in expression were analyzed with Genespring GX. As a result, since expression of the gene group involved in cell proliferation (cell cycle) was confirmed to have decreased in the combination heparanase inhibitor+MMP inhibitor group, while expression in the gene group involved in cell differentiation (keratinization) was confirmed to have increased, the combination of heparanase inhibitor and MMP inhibitor was confirmed to inhibit proliferation of epidermal cells but accelerate their differentiation.

1-2. (canceled)

3. A method for preventing or improving decreases in the barrier function and moisture retention function of skin, comprising the step of applying a combination of one or more matrix metalloproteinase inhibitors and one or more heparanase inhibitors to the skin of a subject in need of the prevention or inhibition of decreases in the barrier function and moisture retention function of skin.
